(19) United States
(12) Patent Application Publication
(10) Pub. No.: US 2008/0268034 A1
(43) Pub. Date: Oct. 30, 2008

(54) Solid Oral Dosage Forms of Ziprasidone Containing Colloidal Silicone Dioxide

(76) Inventors: Girish Karanth, Karnataka (IN); Romi Barat Singh, Uttar Pradesh (IN); Vishnubhotla Nagaprasad, Hyderabad (IN)

Correspondence Address: RANBAXY INC., 600 COLLEGE ROAD EAST, SUITE 2100, PRINCETON, NJ, 08540, US

(21) Appl. No.: 11/813,298

(22) PCT Filed: Jan. 6, 2006
(86) PCT No.: PCT/IB06/00014

§ 371 Date: May 2, 2008

(30) Foreign Application Priority Data
Jan. 7, 2005 (IN) 37/DEL/2005

(51) Int. Cl. A61K 9/48; A61K 9/14; A61K 31/501; A61P 25/18; A61K 9/20

(52) U.S. Cl. 424/451; 424/489; 514/254.02; 424/464

(57) ABSTRACT

The present invention relates to solid oral dosage forms of ziprasidone and salts thereof and processes for their preparation, containing colloidal silicon dioxide.

### TECHNICAL FIELD OF THE INVENTION

The present invention relates to solid oral dosage forms of ziprasidone and salts thereof and processes for their preparation.

### BACKGROUND OF THE INVENTION

Ziprasidone is an antipsychotic used in the treatment of schizophrenia. Chemically, it is 5-[2-[4-(1,2-benzisothiazol-3-yl)-1-piperazinyl]ethyl]-6-chloro-1,3-dihydro-2H-indol-2-one. It is available in the capsule form under the brand name Geodon® sold by Pfizer. The capsules contain ziprasidone hydrochloride, lactose, pregelatinized starch, and magnesium stearate. These are available in dosage strengths of 20, 40, 60 and 80 mg.

Ziprasidone free base and its hydrochloride salt have very poor solubility. In addition to this, ziprasidone is difficult to wet which is problematic when formulating its dosage form. Ziprasidone tends to form agglomerates when it comes in contact with an aqueous liquid. The agglomerates may slow the dissolution of ziprasidone when the dosage form is in contact with gastrointestinal fluids.

U.S. Pat. No. 6,150,366 discloses ziprasidone-containing formulations which use ziprasidone of a mean particle size equal to or less than 85 μm and are shown to exhibit good dissolution properties at physiological pH. The patent also discloses that the rate of dissolution in vitro does not correlate with particle size. Ziprasidone having a particle size of at least at or below 85 μm has a dissolution rate in aqueous media that does not vary substantially with the particle size, and therefore appears to be largely independent of particle size in this range. It is further disclosed that ziprasidone of this particle size can be formulated in a composition which is easily manageable using conventional formulation methodologies and equipment. The patent further discloses that it is not necessary to use extreme measures or specialized technology to maintain relatively tiny particles to facilitate dissolution.

U.S. Pat. No. 4,831,031 discloses the preparation of ziprasidone and salts thereof. By following the process for the production of hydrochloride salt as generally disclosed in the '031 patent, ziprasidone hydrochloride is obtained in a very fine particle size. It would be advantageous to use ziprasidone in this as-obtained fine particle size form for formulating dosage forms as this procedure would reduce at least one process step. However, we have found that ziprasidone of small particle size is fluffy and tends to agglomerate due to surface charge, which decreases the effective available surface area. The decreased exposed surface area results in slowed dissolution of ziprasidone contrary to the expectation that decreased particle size would enhance the solubility. The agglomerates further contribute to handling problems while formulating a dosage form. This also leads to problems of content uniformity in the dosage forms and reproducibility of dissolution profile.

Therefore, the present invention provides a method of preparing solid oral dosage forms of ziprasidone with a fine particle size that exhibit good dissolution properties at physiological pH.

### SUMMARY OF THE INVENTION

In one general aspect there is provided a solid oral dosage form. The solid dosage form includes ziprasidone having a particle size D₉₀ less than or equal to 10 μm, colloidal silicon dioxide in a weight ratio with the ziprasidone of about 1:0.1 to 1:1, and optionally one or more pharmaceutically acceptable excipients.

Embodiments of the dosage form may include one or more of the following features. For example, the ziprasidone may have a D₉₀ less than or equal to 3 μm and the ziprasidone and the colloidal silicon dioxide may be present in a weight ratio of about 1:1.

The one or more pharmaceutically acceptable excipients may include fillers, binders, disintegrants, glidants and lubricants. The filler may be one or more of microcrystalline cellulose, mannitol, sucrose, lactose, dextrose, calcium carbonate and sorbitol. The binder may be one or more of polyvinylpyrrolidone, hydroxypropylcellulose, hydroxypropylmethylcellulose, starch and starch based binders, gelatin and gums. The disintegrant may be one or more crospovidone, croscarmellose sodium, starch, hydroxypropylcellulose, hydroxypropylmethylcellulose, gums and sodium starch glycolate. The lubricant and glidants may be one or more of talc, colloidal silicon dioxide, magnesium stearate, stearic acid and sodium stearyl fumarate.

The solid oral dosage form may be one or more of a tablet, capsule, caplet and granule.

In another general aspect there is provided a process for the preparation of a solid dosage form of ziprasidone. The process includes blending ziprasidone having a particle size D₉₀ of less than or equal to 10 μm with colloidal silicon dioxide in a weight ratio of about 1:0.1 to 1:1 to form a blend; optionally blending the blend with one or more pharmaceutically acceptable excipients; optionally granulating the blend by wet or dry granulation; and formulating the blend into a solid oral dosage form.

Embodiments of the process may include one or more of the following features. For example, the ziprasidone may have a D₉₀ less than or equal to 3 μm or the ziprasidone and the colloidal silicon dioxide may present in a weight ratio of about 1:1

The wet granulation may be done with a binder solution or with solvent. The dry granulation may be done by roller compaction or by slugging.

In another general aspect there is provided a method of treating a psychotic condition in a human. The method includes administering to the human in need thereof a solid oral dosage form comprising ziprasidone having a particle size D₉₀ of less than or equal to 10 μm, colloidal silicon dioxide in a weight ratio with the ziprasidone of about 1:0.1 to 1:1, and optionally one or more pharmaceutically acceptable excipients.

The details of one or more embodiments of the inventions are set forth in the description below. Other features, objects and advantages of the inventions will be apparent from the description and claims.

### DETAILED DESCRIPTION OF THE INVENTION

The term “ziprasidone” as used herein includes ziprasidone free base and pharmaceutically acceptable salts and hydrates thereof. Suitable salts include hydrochloride salt and hydrates thereof. The amount of ziprasidone in the solid oral dosage form is intended to provide a unit therapeutic dose which may be from about 5 mg to about 500 mg; particularly from about 10 mg to about 100 mg.

The notation D₉₀ as used herein means that 90% of the particles have a particle size less than a particular range mentioned. Duo less than 10 μm means 90% of the particles have a particle size less than 10 μm. The particle size analysis has been measured by Malvern light scattering. Ziprasidone particles may have a particle size D₉₀ less than about 10 μm; particularly less than about 5 μm; more particularly less than about 3 μm.

Ziprasidone having these defined particle sizes is found to be very fluffy and tends to form agglomerates, which are difficult to break into unit particles. Without wishing to be bound by theory, the agglomeration of ziprasidone particles may be due to surface charges. Colloidal silicon dioxide, when mixed with these agglomerate, tends to neutralize the surface charges from the particles which then de-segregate into individual particles, with colloidal silicon dioxide acting as a drug carrier. The high surface area of colloidal silicon dioxide is an added advantage in its role as a drug carrier. The ziprasidone-colloidal silicon dioxide mixture can then be easily processed into a desired dosage form following conventional formulation methodology.

Colloidal silicon dioxide is generally used as a glidant to improve the flow of powders while making dosage forms like tablets or granules. Colloidal silicon dioxide is usually added to a final blend prior to compression. It is available under several brand names like AEROSIL® and CAB-O-SIL®. When mixed with ziprasidone, the ratio by weight of ziprasidone to colloidal silicon dioxide is 1:0.1 to 1:1. Within this ratio, it has been found that on increasing the amount of colloidal silicon dioxide with respect to ziprasidone, a graded enhancement in dissolution is observed. Further, increasing the amount of colloidal silicon dioxide to more than 1:1 by weight of ziprasidone results in handling problems due to a significant decrease in bulk density.

In addition to ziprasidone and colloidal silicon dioxide, the solid oral dosage form may contain one or more additional pharmaceutical excipients including fillers, binders, disintegrants, glidants and lubricants.

Suitable fillers include one or more of microcrystalline cellulose, mannitol, sucrose, lactose, dextrose, calcium carbonate, sorbitol and mixtures thereof. The filler may be present in an amount of about 15% to about 80%, particularly from about 30% to about 70% by weight of the solid oral dosage form.

Suitable binders include one or more of polyvinylpyrrolidone, hydroxypropylcellulose, hydroxypropylmethylcellulose, starch and starch based binders, gelatin, gums and mixtures thereof. The binder may be mixed with other excipients or added as a granulating liquid dissolved or dispersed in a suitable solvent. The binder solution/dispersion can be prepared in aqueous or non-aqueous solvents, such as water, ethanol, isopropyl alcohol or mixtures thereof. The binder may be present in an amount of about 0.1% to about 10%, particularly from about 1% to about 5% by weight of the solid oral dosage form.

Suitable disintegrants include one or more of crospovidone, croscarmellose sodium, starch, hydroxypropylcellulose, gums, sodium starch glycolate and mixtures thereof. The disintegrant may be present in an amount of about 1% to about 40%, particularly from about 2% to about 20% by weight of the dosage form.

Suitable lubricants and glidants include talc, colloidal silicon dioxide, magnesium stearate, stearic acid and sodium stearyl fumarate. These may be present in an amount of about 0.1% to about 2% by weight of the dosage form.

The solid oral dosage form as defined herein may be in the form of tablets, caplets and granules that can be filled in capsules or sachets. Particularly suitable are granules as these can be easily processed into other dosage forms like tablets and capsules.

The granules may be prepared by conventional procedures, such as wet granulation and dry granulation. In wet granulation, ziprasidone may be mixed with colloidal silicon dioxide and then mixed with one or more of other pharmaceutical excipients including fillers, binders, disintegrants and granulated with a granulating liquid or a binder solution, followed by drying and sizing the granules. Optionally, the granules may be compressed into tablets using appropriate tooling. Alternatively, the dried granules can be filled into hard gelatin capsules.

Dry granulation may involve mixing ziprasidone with colloidal silicon dioxide. The resultant blend is subsequently transferred to a roller compactor for compaction in a known manner. The roller speed, roller gap width and force of compaction are then adjusted and the blend is fed through the roller compactor. The typical force and other conditions can be easily adjusted by the person skilled in the art. For example, the compaction pressure may be between 25 to 120 bar or typically between 80 to 120 bar. For maintaining the steady output of the compact material from the roller compactor, the rollers may be rotated at a speed of between 1 to 20 rpm, particularly between 2 to 15 rpm or more particularly between 3 to 9 rpm.

When in contact with the counter rotating rollers of the roller compactor, the compression force imparted on the blend by rollers converts the powdered form into a ribbon or compaction sheet. This compact sheet is fed to a mill, such as an oscillating mill, fitted with a screen. The screen can be selected with variable hole diameters depending upon the size of the granules required. After passing through the mill and the screen, the compact is converted into granules of the desired particle size distribution. The granules can also be recompacted to attain desired bulk density and processed again. The granules obtained as above may be filled into capsules or packed in sachets. The granules can also be mixed with one or more of pharmaceutically acceptable excipients and compressed into tablets.

In one aspect of the process, direct compression may be used for preparing a tablet by mixing ziprasidone and colloidal silicon dioxide. The blend is further mixed with one or more fillers, binders, disintegrants, lubricants and glidants, and compressed into tablets using appropriate tooling.

In one embodiment, the solid oral dosage form of ziprasidone may be prepared by blending ziprasidone and colloidal silicon dioxide in a non-shear blender; lubricating the above blend with a lubricant; compacting the blend using a roller compactor; milling the compacts into granules of appropriate size using an oscillating granulator; lubricating the sized granules with a lubricant and filling into hard gelatin capsules.

In another embodiment, the solid oral dosage form of ziprasidone may be prepared by blending ziprasidone and colloidal silicon dioxide in a non-shear blender; lubricating the above blend with a lubricant; compacting the blend using a roller compactor; milling the compacts into granules of appropriate size using an oscillating granulator; mixing the sized granules with filler and further lubricating the blend with a lubricant and filling into hard gelatin capsules.

The solid oral dosage form of ziprasidone may also be prepared by blending ziprasidone and colloidal silicon dioxide in a suitable blender; optionally mixing one or more of pharmaceutically acceptable excipients; and granulating with a binder solution; drying the granules; lubricating the dried granules and filling into hard gelatin capsules.

In another embodiment, the solid oral dosage form of ziprasidone may be prepared by blending ziprasidone and colloidal silicon dioxide in a suitable blender and granulating with a binder solution; drying the granules; mixing the sized granules with filler; lubricating the blend with a lubricant; and filling into hard gelatin capsules.

In one embodiment, the solid oral dosage form of ziprasidone may be prepared by blending ziprasidone and colloidal silicon dioxide in a non-shear blender; lubricating the above blend with a lubricant; compacting the blend using a roller compactor; milling the compacts into granules of appropriate size using an oscillating granulator; lubricating the sized granules with a lubricant and compressing into tablets using appropriate tooling.

In another embodiment, the solid oral dosage form of ziprasidone may be prepared by blending ziprasidone and colloidal silicon dioxide in a non-shear blender; lubricating the above blend with a lubricant; compacting the blend using a roller compactor; milling the compacts into granules of appropriate size using oscillating granulator; mixing the sized granules with one or more of filler, disintegrant and glidant, lubricating the blend with a lubricant, and compressing into tablets.

In another embodiment, the solid oral dosage form of ziprasidone may be prepared by blending ziprasidone and colloidal silicon dioxide in a suitable blender and granulating with a binder solution; drying the granules; mixing the sized granules with one or more of fillers, disintegrants, glidants; lubricating the blend with a lubricant; and compressing into tablets using appropriate tooling.

In yet another embodiment, the solid oral dosage form of ziprasidone may be prepared by blending ziprasidone and colloidal silicon dioxide in a suitable blender; lubricating the above blend with a lubricant; compacting the blend by slugging; milling the compacts into granules of appropriate size using an oscillating granulator; lubricating the sized granules with a lubricant; and filling into hard gelatin capsules.

The invention described herein is further illustrated by the following examples but these should not be construed as limiting the scope of the invention.

| | EXAMPLES | |
|---|---|---|
| | Quantity (mg) | |
| Ingredient | Comparative Example | Example 1 |
| Ziprasidone | 43.747 | 43.747 |
| Hydrochloride | (D₉₀ 16 μm) | (D₉₀ 3 μm) |
| (eq. to ziprasidone base | | |
| 40 mg) | | |
| Magnesium stearate | 0.750 | 0.750 |
| Total | 44.497 | 44.497 |

### Procedure:

Ziprasidone hydrochloride and magnesium stearate were blended in a non-shear blender. The blend was compacted with a roller compactor. The compacts were milled into granules using an oscillating granulator. The granules were recompacted with a roller compactor and the compacts thus formed were milled using a Quadro Comill. The granules were filled in hard gelatin capsules.

| | EXAMPLES 2-4 | | |
|---|---|---|---|
| | Quantity (mg) | | |
| Ingredient | Example 2 | Example 3 | Example 4 |
| Ziprasidone | 43.747* | 43.747* | 43.747* |
| Hydrochloride | (D₉₀ 3 μm) | (D₉₀ 3 μm) | (D₉₀ 3 μm) |
| (eq. to ziprasidone base | | | |
| 40 mg) | | | |
| Colloidal silicon dioxide | 10 | 20 | 40 |
| Magnesium stearate | 0.750 | 0.750 | 0.750 |
| Total | 54.497 | 64.497 | 84.497 |
| Ziprasidone:Colloidal | 1:0.25 | 1:0.5 | 1:1 |
| silicon dioxide (W/W) | | | |

*equivalent to Ziprasidone base after potency and moisture adjustment.

### Procedure:

Ziprasidone hydrochloride and colloidal silicon dioxide were sifted and blended in a non-shear blender. The blend was lubricated with magnesium stearate. The lubricated blend was compacted using a roller compactor. The compacts were milled into granules using an oscillating granulator. The granules were recompacted with a roller compactor and the compacts thus formed were milled into granules of appropriate size using a Quadro Comill. The granules were filled into hard gelatin capsules.

Alternatively, Examples 2-4 may have extragranular excipients, either compacted or uncompacted, for improvement in flow and other process parameters. An example to this effect is given below as Example 5.

EXAMPLE 5

| | |
|---|---|
| Ingredient | Quantity (mg) |

Intragranular

| | |
|---|---|
| Ziprasidone HCl (Equivalent to | 43.52 |
| 40 mg of Ziprasidone) | (D₉₀ 3 μm) |
| Colloidal silicon dioxide | 19.98 |
| Magnesium stearate | 0.50 |
| Total | 64.00 |

Extragmnular

| | |
|---|---|
| Lactose monohydrate | 19.00 |
| Microcrystalline cellulose | 65.75 |
| Magnesium stearate | 1.25 |
| Total | 150.00 |
| Ziprasidone:Colloidal silicon | 1:0.5 |
| dioxide (W/W) | |

### Procedure:

Ziprasidone hydrochloride and colloidal silicon dioxide were sifted and blended in a non-shear blender. The blend was lubricated with magnesium stearate. The lubricated blend was compacted using a roller compactor. The compacts were milled into granules using an oscillating granulator. The granules were recompacted with a roller compactor and the compacts thus formed were milled into granules of appropriate size using a Quadro Comill. Separately, lactose and a portion of microcrystalline cellulose were blended in a non-shear blender and lubricated with magnesium stearate. The lubricated blend was compacted using a roller compactor. The compacts were milled into granules using an oscillating granulator. The granules were mixed with the ziprasidone-containing granules, blended with the remaining microcrystalline cellulose and magnesium stearate, and filled into hard gelatin capsules.

TABLE 1
Dissolution profiles of the capsules as measured in a USP
type II dissolution apparatus, at 75 rpm in 900 ml of 0.05M
Phosphate buffer with 2% sodium lauryl sulphate.

| | % Drug Release | | | | | |
|---|---|---|---|---|---|---|
| | Compara- | Exam- | Exam- | Exam- | Exam- | Exam- |
| Time | tive | ple | ple | ple | ple | ple |
| (min) | Example | 1 | 2 | 3 | 4 | 5 |
| 10 | — | — | — | — | — | 34 |
| 20 | — | — | — | — | — | 49 |
| 30 | 63 | — | 38 | 61 | 69 | 61 |
| 45 | 67 | — | 46 | 66 | 73 | 66 |
| 60 | — | 43 | 52 | 71 | 77 | 71 |
| ∞ | 83 | 47 | 70 | 81 | 85 | — |

As can be seen from the comparative example and Example 1 in Table 1, in which ziprasidone hydrochloride has a particle size D₉₀ of 16 μm and 3 μm, respectively, there is an appreciable decrease in the percentage drug release of ziprasidone hydrochloride in spite of the use of a finer particle size. However, from Examples 2-5, it can be appreciated that the dissolution is markedly improved when colloidal silicon dioxide is mixed with ziprasidone hydrochloride.

While the present invention has been described in terms of its specific embodiments, certain modifications and equivalents will be apparent to those skilled in the art and are included within the scope of the present invention.

We claim:

1. A solid oral dosage form comprising ziprasidone having a particle size D₉₀ less than or equal to 10 μm, colloidal silicon dioxide in a weight ratio with the ziprasidone of about 1:0.1 to 1:1, and optionally one or more pharmaceutically acceptable excipients.

2. The solid dosage form according to claim 2, wherein the ziprasidone has a D₉₀ of less than or equal to 3 μm.

3. The solid dosage form according to claim 1, wherein the ziprasidone and the colloidal silicon dioxide are present in a weight ratio of about 1:1.

4. (canceled)

5. (canceled)

6. (canceled)

7. (canceled)

8. (canceled)

9. The solid oral dosage form according claim 1, wherein the solid oral dosage form comprises one or more of a tablet, capsule, caplet and granule.

10. A process for the preparation of a solid dosage form of ziprasidone, the process comprising:
a) blending ziprasidone having a particle size D₉₀ of less than or equal to 10 μm with colloidal silicon dioxide in a weight ratio of about 1:0.1 to 1:1 to form a blend;
b) optionally blending the blend with one or more pharmaceutically acceptable excipients;
c) optionally granulating the blend by wet or dry granulation; and
d) formulating the blend into a solid oral dosage form.

11. The process according to claim 10, wherein the ziprasidone has a D₉₀ of less than or equal to 3 μm.

12. (canceled)

13. The process according to claim 10, wherein the wet granulation comprises a binder solution or a solvent.

14. The process according to claim 10, wherein the dry granulation comprises roller compaction.

15. The process according to claim 10, wherein the dry granulation comprises slugging.

16. A method of treating a psychotic condition in a human wherein the method comprises administering to the human in need thereof a solid oral dosage form comprising ziprasidone having a particle size D₉₀ of less than or equal to 10 μm, colloidal silicon dioxide in a weight ratio with the ziprasidone of about 1:0.1 to 1:1 and optionally one or more pharmaceutically acceptable excipients.
